(19) United States
(12) Patent Application Publication
(10) Pub. No.: US 2013/0253600 A1
(43) Pub. Date: Sep. 26, 2013

(54) INTERNET SUPPORTED SOFTWARE UPDATES FOR MEDICAL DEVICES

(71) Applicant: PHYSIO-CONTROL, INC., Redmond, WA (US)

(72) Inventors: Kevin C. Drew, Snohomish, WA (US); Dana S. Lewis, Woodinville, WA (US)

(73) Assignee: PHYSIO-CONTROL, INC., Redmond, WA (US)

(21) Appl. No.: 13/629,426

(22) Filed: Sep. 27, 2012

Related U.S. Application Data
(60) Provisional application No. 61/615,800, filed on Mar. 26, 2012.

(51) Int. Cl. G06F 9/44; A61N 1/39

(52) U.S. Cl. 607/5; 717/173

(57) ABSTRACT

A computer can include a first communication port for establishing a first connection with a medical device, a second communication port for establishing a second connection with a network, and an agent configured to query a collection of device records within the network to determine whether a device record specific to the medical device exists. The computer can also include a processor configured to receive over the second connection a message from the network responsive to the query, the message including an upgrade link specific to a device type corresponding to the device. The processor can also be configured to send to the medical device over the first connection an upgrade of a software application on the medical device responsive to a user selecting the upgrade link, where the software application is structured to cause the device to operate.

### PRIORITY CLAIM

This application claims the benefit of U.S. Provisional Application No. 61/615,800, filed Mar. 26, 2012 and titled INTERNET SUPPORTED SOFTWARE UPDATES FOR MEDICAL DEVICES, the content of which is hereby fully incorporated by reference herein.

### FIELD

This invention generally relates to medical devices, such as external defibrillators.

### BACKGROUND

In humans, the heart beats to sustain life. In normal operation, it pumps blood through the various parts of the body. More particularly, the various chamber of the heart contract and expand in a periodic and coordinated fashion, which causes the blood to be pumped regularly. More specifically, the right atrium sends deoxygenated blood into the right ventricle. The right ventricle pumps the blood to the lungs, where it becomes oxygenated, and from where it returns to the left atrium. The left atrium pumps the oxygenated blood to the left ventricle. The left ventricle, then, expels the blood, forcing it to circulate to the various parts of the body.

The heart chambers pump because of the heart's electrical control system. More particularly, the sinoatrial (SA) node generates an electrical impulse, which generates further electrical signals. These further signals cause the above-described contractions of the various chambers in the heart, in the correct sequence. The electrical pattern created by the sinoatrial (SA) node is called a sinus rhythm.

Sometimes, however, the electrical control system of the heart malfunctions, which can cause the heart to beat irregularly, or not at all. The cardiac rhythm is then generally called an arrhythmia. Arrhythmias may be caused by electrical activity from locations in the heart other than the SA node. Some types of arrhythmia may result in inadequate blood flow, thus reducing the amount of blood pumped to the various parts of the body. Some arrhythmias may even result in a Sudden Cardiac Arrest (SCA). In a SCA, the heart fails to pump blood effectively, and, if not treated, death can occur. In fact, it is estimated that SCA results in more than 250,000 deaths per year in the United States alone. Further, a SCA may result from a condition other than an arrhythmia.

One type of arrhythmia associated with SCA is known as Ventricular Fibrillation (VF). VF is a type of malfunction where the ventricles make rapid, uncoordinated movements, instead of the normal contractions. When that happens, the heart does not pump enough blood to deliver enough oxygen to the vital organs. The person's condition will deteriorate rapidly and, if not reversed in time, they will die soon, e.g. within ten minutes.

Ventricular Fibrillation can often be reversed using a life-saving device called a defibrillator. A defibrillator, if applied properly, can administer an electrical shock to the heart. The shock may terminate the VF, thus giving the heart the opportunity to resume pumping blood. If VF is not terminated, the shock may be repeated, often at escalating energies.

A challenge with defibrillation is that the electrical shock must be administered very soon after the onset of VF. There is not much time: the survival rate of persons suffering from VF decreases by about 10% for each minute the administration of a defibrillation shock is delayed. After about 10 minutes the rate of survival for SCA victims averages less than 2%.

The challenge of defibrillating early after the onset of VF is being met in a number of ways. First, for some people who are considered to be at a higher risk of VF or other heart arrhythmias, an Implantable Cardioverter Defibrillator (ICD) can be implanted surgically. An ICD can monitor the person's heart, and administer an electrical shock as needed. As such, an ICD reduces the need to have the higher-risk person be monitored constantly by medical personnel.

Regardless, VF can occur unpredictably, even to a person who is not considered at risk. As such, VF can be experienced by many people who lack the benefit of ICD therapy. When VF occurs to a person who does not have an ICD, they collapse, because blood flow has stopped. They should receive therapy quickly.

For a VF victim without an ICD, a different type of defibrillator can be used, which is called an external defibrillator. External defibrillators have been made portable, so they can be brought to a potential VF victim quickly enough to revive them.

During VF, the person's condition deteriorates, because the blood is not flowing to the brain, heart, lungs, and other organs. Blood flow must be restored, if resuscitation attempts are to be successful.

Cardiopulmonary Resuscitation (CPR) is one method of forcing blood flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. As such, CPR can be beneficial for persons experiencing VF, because it slows the deterioration that would otherwise occur while a defibrillator is being retrieved. Indeed, for patients with an extended down-time, survival rates are higher if CPR is administered prior to defibrillation.

Advanced medical devices can actually coach a rescuer who performs CPR. For example, a medical device can issue instructions, and even prompts, for the rescuer to perform CPR more effectively.

### BRIEF SUMMARY

The present description gives instances of devices, systems, software and methods, the use of which may help overcome problems and limitations of the prior art.

In one embodiment, a computer may include a first communication port for establishing a first connection with a medical device, a second communication port for establishing a second connection with a network, and an agent configured to query a collection of device records within the network to determine whether a device record specific to the medical device exists. The computer may also include a processor configured to receive over the second connection a message from the network that includes an upgrade link specific to a device type corresponding to the device. The processor may send to the medical device over the first connection an upgrade of a software application on the medical device responsive to a user selecting the upgrade link. The software application may be structured to cause the device to operate.

In another embodiment, a medical device may include an external defibrillator having a housing, an energy storage module within the housing for storing an electrical charge, a defibrillation port for guiding via electrodes the stored electrical charge to a person, a communication port within the housing for establishing a connection with a computer in communication with a network, and a memory within the housing for storing a software application structured to cause the device to operate. The device may also include a processor within the housing configured to execute the software application and to implement an upgrade of the software application received from the computer over the connection responsive to a user selecting a link in a message received by the computer from the network. The link may be specific to the device type corresponding to the device.

An advantage over the prior art is that a software application on a medical device, such as an external defibrillator, may be efficiently and easily upgraded responsive to a user selecting an upgrade link in a message. The message containing the upgrade link may be sent using a network in a number of situations, such as when a purchaser places an order for the medical device or when a purchaser places an order for another medical device having a type that is at least substantially similar to the medical device. The network may be Internet based and the upgrade link may be sent over the network in an electronic communication, such as an email message, thus providing a user increased flexibility with regard to the upgrade. For example, the user may direct the upgrade to occur even if the device is at a location that is remote from the user.

These and other features and advantages of this description will become more readily apparent from the following Detailed Description, which proceeds with reference to the drawings, in which:

### BRIEF DESCRIPTION OF THE DRAWINGS

FIG. 1 is a diagram of a scene where an external defibrillator is used to save the life of a person according to embodiments.

FIG. 2 is a table listing two main types of the external defibrillator shown in FIG. 1, and who they might be used by.

FIG. 3 is a functional block diagram showing components of an external defibrillator, such as the one shown in FIG. 1, which is made according to embodiments.

FIG. 4 is a functional block diagram showing components of an example network-based system including a medical device according to embodiments.

FIG. 5 is a flowchart for illustrating example methods of a medical device interacting with a network-based system, such as the one shown in FIG. 4, according to embodiments.

### DETAILED DESCRIPTION

As has been mentioned, the present description is about medical devices, methods of operating such medical devices, and a programmed processor to control such medical devices for controlling enabling features of the medical device based on an upgrade link that may be sent in an electronic message.

Embodiments are now described in more detail.

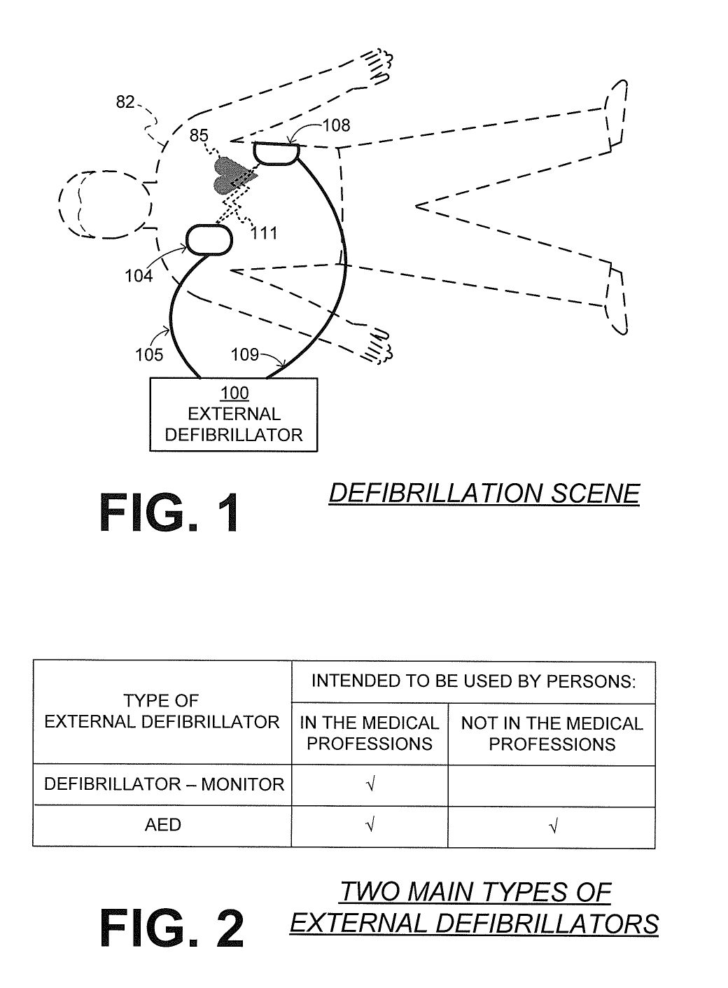

FIG. 1 is a diagram of a defibrillation scene. A person 82 is lying on their back. Person 82 could be a patient in a hospital, or someone found unconscious, and then turned to be on their back. Person 82 is experiencing a condition in their heart 85, which could be Ventricular Fibrillation (VF).

A portable external defibrillator 100 has been brought close to person 82. At least two defibrillation electrodes 104, 108 are usually provided with external defibrillator 100, and are sometimes called electrodes 104, 108. Electrodes 104, 108 are coupled with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, goes also through heart 85, in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing two main types of external defibrillators, and who they are primarily intended to be used by. A first type of defibrillator 100 is generally called a defibrillator-monitor, because it is typically formed as a single unit in combination with a patient monitor. A defibrillator-monitor is sometimes called monitor-defibrillator. A defibrillator-monitor is intended to be used by persons in the medical professions, such as doctors, nurses, paramedics, emergency medical technicians, etc. Such a defibrillator-monitor is intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Indeed, the people who will first reach the VF sufferer may not be in the medical professions.

Increasing awareness has resulted in AEDs being deployed in public or semi-public spaces, so that even a member of the public can use one, if they have obtained first aid and CPR/AED training on their own initiative. This way, defibrillation can be administered soon enough after the onset of VF, to hopefully be effective in rescuing the person.

There are additional types of external defibrillators, which are not listed in FIG. 2. For example, a hybrid defibrillator can have aspects of an AED, and also of a defibrillator-monitor. A usual such aspect is additional ECG monitoring capability.

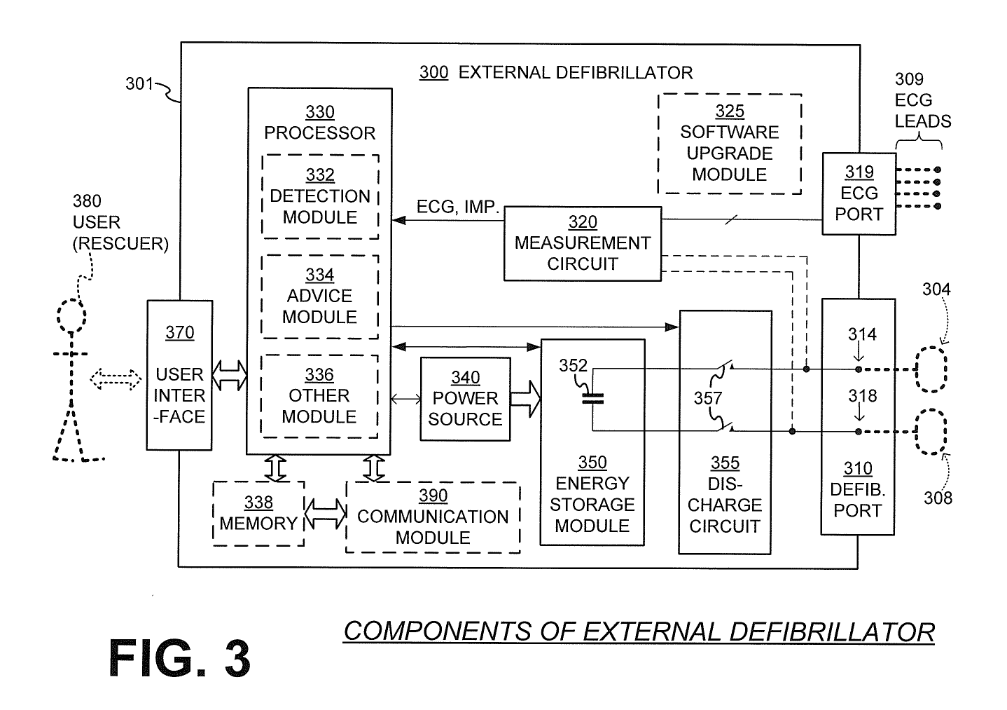

FIG. 3 is a diagram showing components of an external defibrillator 300 made according to embodiments. These components can be, for example, in external defibrillator 100 of FIG. 1. Plus, these components of FIG. 3 can be provided in a housing 301, which is also known as casing 301.

External defibrillator 300 is intended for use by a user 380, who would be the rescuer. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding via electrodes to person 82 an electrical charge that has been stored in defibrillator 300, as will be seen later in this document.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or from a different number of leads. Moreover, a defibrillator-monitor could have additional ports (not shown), and a software upgrade module 325 configured to cause a software application pertaining to the defibrillator 300 to be upgraded responsive to certain conditions and/or events.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions. In addition, if other component 325 is indeed provided, it may be operated in part by processor 330, etc.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. The programs can be operational for the inherent needs of processor 330, and can also include protocols and ways that decisions can be made by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. Sometimes, a combination is used, of rechargeable and non-rechargeable battery packs. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, and so on.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and so on.

Defibrillator 300 further includes a user interface 370 for user 380. User interface 370 can be made in any number of ways. For example, interface 370 may include a screen, to display what is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, incident information, therapy attempted, CPR performance, and so on.

A feature of a defibrillator can be CPR-prompting. Prompts are issued to the user, visual or by sound, so that the user can administer CPR. Examples are taught in U.S. Pat. No. 6,334,070 and No. 6,356,785.

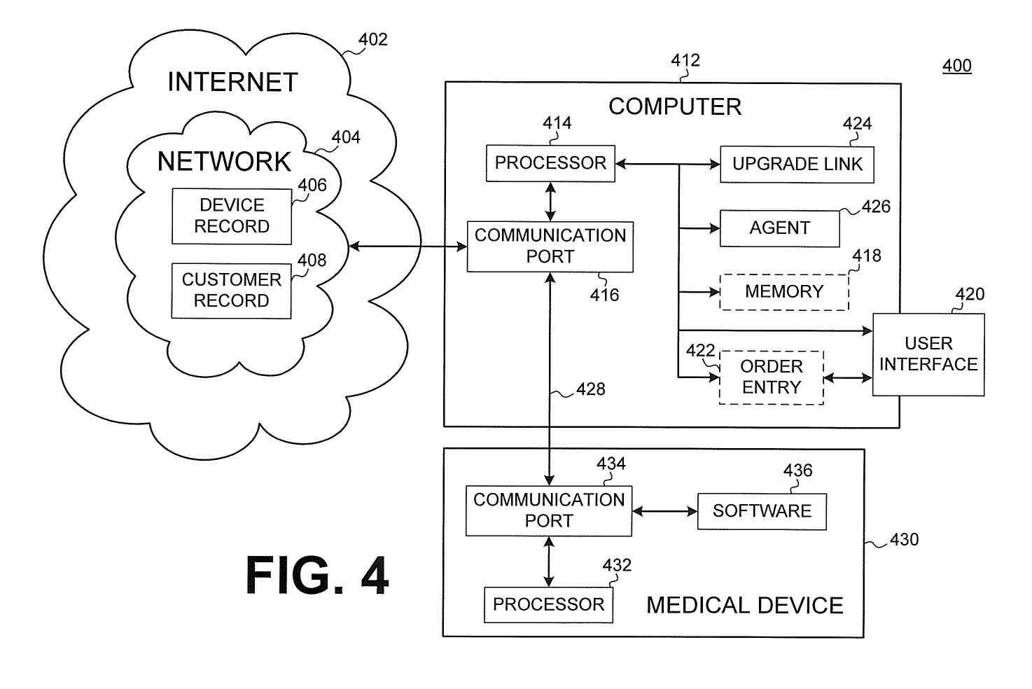

FIG. 4 is a functional block diagram showing components of a system 400 having a network 404, a computer 412, and a medical device 430, such as an external defibrillator, according to certain embodiments. Network 404 may be based on the Internet 402 and may be configured to store a device record 406 corresponding to a medical device, such as the medical device 430. The device record 406 may include a device type corresponding to the medical device 430, for example. Network 404 may also be configured to store a customer record 408 corresponding to a medical device, such as medical device 430, another medical device, or both. Either or both of the device record 406 and customer record 408 may be stored by a storage device such as a database, for example.

Computer 412 includes a communication port 416 for communicating with the network 404 and the medical device 430. In certain embodiments, the communication port 416 may include a separate first communication port for establishing a first connection with the medical device 430 and a separate second communication port for establishing a second connection with the network 404.

An agent 426 on the computer 412 may be configured to query a collection of device records, e.g., including device record 406, within the network 404 to determine whether a device record specific to the medical device 430 exists.

Computer 412 further includes a processor 414 configured to receive a message from the network 404 responsive to the query, the message including an upgrade link 424 that is specific to a device type corresponding to the device 430. The message may also include credentials pertaining to the medical device 430, the upgrade, or both the medical device 430 and the upgrade, and instructions for implementing the upgrade. The message may be stored by a memory 418 of, or otherwise connectable with, the computer 412.

The upgrade link 424 may be at least partially based on a device record 406 within the network 404 that is specific to the medical device 430. For example, the upgrade link 424 may be specific to a device type corresponding to the medical device 430. Alternatively or in addition thereto, the upgrade link 424 may be at least partially based on a customer record 408 that is stored within the network 404 and corresponds to the medical device 430.

In certain embodiments, the message is received from the network 404 responsive to a purchaser placing an order for the medical device 430. For example, the purchaser may interact with a user interface 420 of the computer 412 to place the order using an order entry module or application 422.

In alternative embodiments, the message is received from the network 404 responsive to a purchaser placing an order for another medical device having a type that is at least substantially similar to the medical device 430. In these embodiments, the order may include a customer account number, account contact credentials, and a listing of at least one medical device for which upgrades are available, will become available, or both are available and will become available. Alternatively or in addition thereto, the order may specify at least one upgrade version to be applied to the software application 436. Alternatively or in addition thereto, the order may specify at least one configuration setting of the medical device 430.

As indicated by 428, the processor 414 can send to the medical device 430 an upgrade of a software application 436 on the device 430 responsive to a user selecting the upgrade link 424, the software application 436 being configured to cause the device 430 to operate. Sending the upgrade of the software application 436 may include sending an upgrade application to the medical device 430 over the connection 428 with the computer 412.

Medical device 430 includes a communication port 434 for establishing the connection with the computer 412, as indicated by 428. Device 430 also includes a processor 432 configured to execute the software application 436 and to implement an upgrade of the software application 436 as received from the computer 412 over the connection 428 responsive to a user selecting the upgrade link 424, which is specific to the device type corresponding to the device 430. The processor 432 may also be configured to provide a notification to cause the communication port 434 to establish the connection 428 with the computer 412.

In certain embodiments, the medical device 430 may be a defibrillator that includes a housing, an energy storage module within the housing for storing an electrical charge, a defibrillation port for guiding via electrodes the stored electrical charge to a person, and a memory within the housing. The memory may store the software application 436, for example. The defibrillator may also include a user interface structured to deliver prompts to a user during a defibrillation session, for example.

The functions of this description may be implemented by one or more devices that include logic circuitry. The device performs functions and/or methods as are described in this document. The logic circuitry may include a processor that may be programmable for a general purpose, or dedicated, such as microcontroller, a microprocessor, a Digital Signal Processor (DSP), etc. For example, the device may be a digital computer like device, such as a general-purpose computer selectively activated or reconfigured by a computer program stored in the computer. Alternately, the device may be implemented by an Application Specific Integrated Circuit (ASIC), etc.

Moreover, methods are described below. The methods and algorithms presented herein are not necessarily inherently associated with any particular computer or other apparatus. Rather, various general-purpose machines may be used with programs in accordance with the teachings herein, or it may prove more convenient to construct more specialized apparatus to perform the required method steps. The required structure for a variety of these machines will become apparent from this description.

In all cases there should be borne in mind the distinction between methods in this description, and the method of operating a computing machine. This description relates both to methods in general, and also to steps for operating a computer and for processing electrical or other physical signals to generate other desired physical signals.

Programs are additionally included in this description, as are methods of operation of the programs. A program is generally defined as a group of steps leading to a desired result, due to their nature and their sequence. A program is usually advantageously implemented as a program for a computing machine, such as a general-purpose computer, a special purpose computer, a microprocessor, etc.

Storage media are additionally included in this description. Such media, individually or in combination with others, have stored thereon instructions of a program made according to the invention. A storage medium according to the invention is a computer-readable medium, such as a memory, and is read by the computing machine mentioned above.

Performing the steps or instructions of a program requires physical manipulations of physical quantities. Usually, though not necessarily, these quantities may be transferred, combined, compared, and otherwise manipulated or processed according to the instructions, and they may also be stored in a computer-readable medium. These quantities include, for example electrical, magnetic, and electromagnetic signals, and also states of matter that can be queried by such signals. It is convenient at times, principally for reasons of common usage, to refer to these quantities as bits, data bits, samples, values, symbols, characters, images, terms, numbers, or the like. It should be borne in mind, however, that all of these and similar terms are associated with the appropriate physical quantities, and that these terms are merely convenient labels applied to these physical quantities, individually or in groups.

This detailed description is presented largely in terms of flowcharts, display images, algorithms, and symbolic representations of operations of data bits within at least one computer readable medium, such as a memory. Indeed, such descriptions and representations are the type of convenient labels used by those skilled in programming and/or the data processing arts to effectively convey the substance of their work to others skilled in the art. A person skilled in the art of programming may use these descriptions to readily generate specific instructions for implementing a program according to the present invention.

Often, for the sake of convenience only, it is preferred to implement and describe a program as various interconnected distinct software modules or features, individually and collectively also known as software. This is not necessary, however, and there may be cases where modules are equivalently aggregated into a single program with unclear boundaries. In any event, the software modules or features of this description may be implemented by themselves, or in combination with others. Even though it is said that the program may be stored in a computer-readable medium, it should be clear to a person skilled in the art that it need not be a single memory, or even a single machine. Various portions, modules or features of it may reside in separate memories, or even separate machines. The separate machines may be connected directly, or through a network, such as a local access network (LAN), or a global network, such as the Internet.

It will be appreciated that some of these methods may include software steps that may be performed by different modules of an overall software architecture. For example, data forwarding in a router may be performed in a data plane, which consults a local routing table. Collection of performance data may also be performed in a data plane. The performance data may be processed in a control plane, which accordingly may update the local routing table, in addition to neighboring ones. A person skilled in the art will discern which step is best performed in which plane.

An economy is achieved in the present document in that a single set of flowcharts is used to describe both programs, and also methods. So, while flowcharts are described in terms of boxes, they can mean both method and programs.

For this description, the methods may be implemented by machine operations. In other words, embodiments of programs are made such that they perform methods of the invention that are described in this document. These may be optionally performed in conjunction with one or more human operators performing some, but not all of them. As per the above, the users need not be collocated with each other, but each only with a machine that houses a portion of the program. Alternately, some of these machines may operate automatically, without users and/or independently from each other.

Methods are now described.

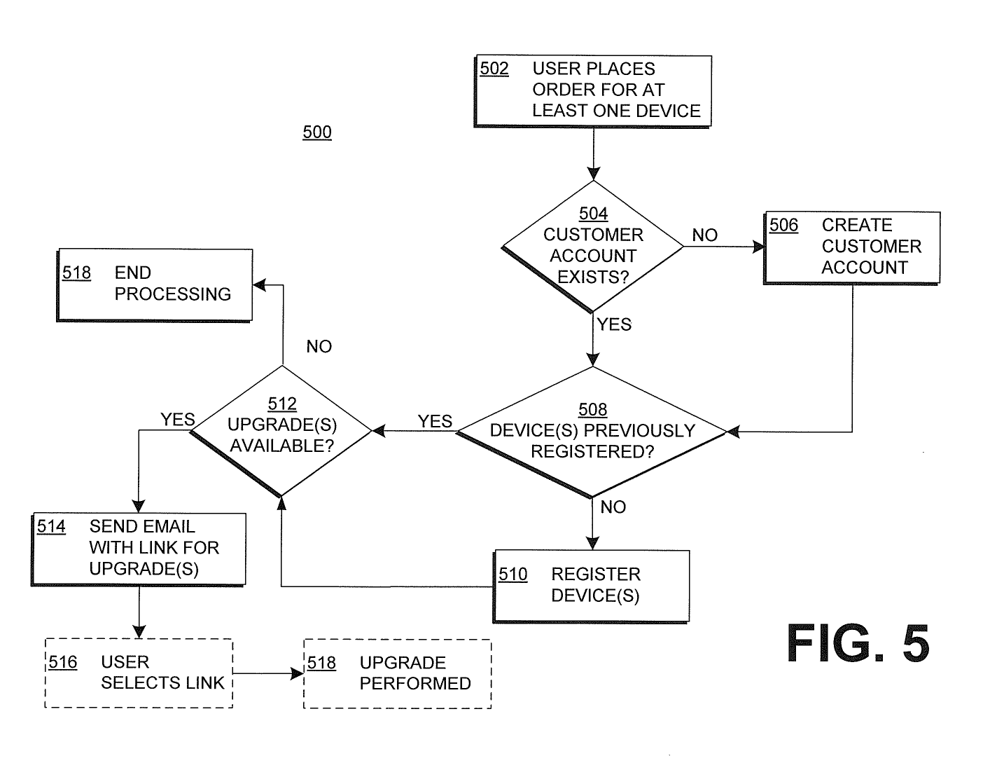

FIG. 5 shows a flowchart 500 for describing methods according to embodiments. According to an operation 502, a user or purchaser places an order for at least one medical device.

According to a next operation 504, a determination is made as to whether a customer account already exists so that the device may be associated with a customer account. For example, a network may determine whether a customer account pertaining to the purchaser of the medical device exists and, if so, the network may access an existing customer account; otherwise, the network may create a new customer account, as indicated by 506. In so doing, the network may establish an expiration date for the new customer account, where the expiration date specifies a time limit in which any upgrade is to be applied to a software application on the device.

According to a next operation 508, a determination is made as to whether the device was previously registered. If not, the network may register the device, as indicated by 510; otherwise, a determination is made as to whether an upgrade is available for a software application specific to the medical device, as indicated by 512. If not, processing ends, as indicated by 518; otherwise, the method may proceed to 514.

According to the operation at 514, the network sends to a computer an email message that includes a link to an upgrade application configured to implement the upgrade for the software application.

According to an optional next operation at 516, a user selects the upgrade link in the email message. Responsive thereto, the upgrade may be performed, as indicated by 518. For example, a processor of the device may be configured to implement the upgrade by executing an upgrade application received from the computer over a connection therewith. In certain embodiments, the upgrade application may be automatically downloaded and executed responsive to the user selecting the link in the email message at 516.

In certain embodiments, a processor of the medical device is configured to delete the upgrade application after the upgrade has been implemented. Alternatively or in addition thereto, the processor may be further configured to execute a self-test of the medical device after the upgrade has been implemented. The processor may be configured to provide a notification that the self-test was successful. Alternatively or in addition thereto, the processor may be configured to delete the upgrade application responsive to an indication that the self-test was successful.

In this description, numerous details have been set forth in order to provide a thorough understanding. In other instances, well-known features have not been described in detail in order to not obscure unnecessarily the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems.

The following claims define certain combinations and subcombinations of elements, features, steps, and/or functions, which are regarded as novel and non-obvious. Additional claims for other combinations and subcombinations may be presented in this or a related document.

What is claimed is:

1. A computer, comprising:
a first communication port for establishing a first connection with a medical device;
a second communication port for establishing a second connection with a network;
an agent configured to query a collection of device records within the network to determine whether a device record specific to the medical device exists; and
a processor configured to receive over the second connection a message from the network responsive to the query, the message including an upgrade link specific to a device type corresponding to the device, and the processor also configured to send to the medical device over the first connection an upgrade of a software application on the medical device responsive to a user selecting the upgrade link, the software application structured to cause the device to operate.

2. The computer of claim 1, in which
the upgrade link is at least partially based on the device record within the network that is specific to the medical device.

3. The computer of claim 1, in which
the upgrade link is at least partially based on a model number of the medical device.

4. The computer of claim 1, in which
the upgrade link is at least partially based on a customer record that is stored within the network and corresponds to the medical device.

5. The computer of claim 1, in which
the message is received from the network responsive to a purchaser placing an order for the medical device.

6. The computer of claim 1, in which
the message is received from the network responsive to a purchaser placing an order for purchasing another medical device having a type that is at least substantially similar to the device type corresponding to the device.

7. The computer of claim 6, in which
the order includes a customer account number, account contact credentials, and a listing of at least one medical device for which upgrades are available, will become available, or both are available and will become available.

8. The computer of claim 7, in which
the order specifies at least one upgrade version to be applied to the software application.

9. The computer of claim 7, in which
the order specifies at least one configuration setting of the medical device.

10. The computer of claim 6, in which
the network determines whether a customer account pertaining to a purchaser of the medical device exists.

11. The computer of claim 10, in which
the network accesses an existing customer account.

12. The computer of claim 10, in which
the network creates a new customer account.

13. The computer of claim 12, in which
the network establishes an expiration date for the new customer account.

14. The computer of claim 13, in which
the expiration date specifies a time limit in which any upgrade is to be applied to the software application.

15. A medical device, the medical device comprising:
a housing;
an energy storage module within the housing for storing an electrical charge;
a defibrillation port for guiding via electrodes the stored electrical charge to a person;
a communication port within the housing for establishing a connection with a computer in communication with a network;
a memory within the housing for storing a software application structured to cause the device to operate; and
a processor within the housing configured to execute the software application and to implement an upgrade of the software application received from the computer over the connection responsive to a user selecting a link in a message received by the computer from the network, the link specific to a device type corresponding to the device.

16. The medical device of claim 15, in which
the processor is configured to implement the upgrade by executing an upgrade application received from the computer over the connection.

17. The medical device of claim 16, in which
the processor is configured to delete the upgrade application after the upgrade has been implemented.

18. The medical device of claim 15, in which
the processor is further configured to execute a self-test of the medical device after the upgrade has been implemented.

19. The medical device of claim 18, in which
the processor is configured to provide a notification that the self-test was successful.

20. The medical device of claim 18, in which
the processor is configured to delete the upgrade application responsive to an indication that the self-test was successful.

21. The medical device of claim 15, in which
the email message further includes credentials pertaining to the medical device, the upgrade, or both the medical device and the upgrade, and instructions for implementing the upgrade.

22. A method, comprising:
receiving an order for at least one medical device;
associating the order with a customer account;
registering the medical device;
determining whether an upgrade is available for a software application specific to the medical device; and
sending an email message that includes a link to an upgrade application configured to implement the upgrade for the software application.

23. The method of claim 22, further comprising:
determining whether the customer account exists.

24. The method of claim 23, further comprising:
responsive to a determination that the customer account does not exist, creating the customer account.

25. The method of claim 24, further comprising:
establishing an expiration date for the customer account, the expiration date specifying a time limit in which any upgrade is to be applied to the software application.

26. The method of claim 22, further comprising:
automatically downloading and executing the upgrade application responsive to a user selecting the link in the email message.

27. The method of claim 26, further comprising:
deleting the upgrade application after the upgrade has been implemented.

28. The method of claim 26, further comprising:
executing a self-test of the medical device after the upgrade has been implemented.

29. The method of claim 28, further comprising:
providing a notification that the self-test was successful.

30. The method of claim 28, further comprising:
deleting the upgrade application responsive to an indication that the self-test was successful.
